(19) United States
(12) Patent Application Publication
(10) Pub. No.: US 2026/0334843 A1
(43) Pub. Date: Oct. 29, 2026

(54) ELECTROPHYSIOLOGY CATHETER

(71) Applicant: Boston Scientific Scimed, Inc., Maple Groe, MN (US)

(72) Inventors: Ryan Alan Kleve, Golden Valley, MN (US); Allan Charles Shuros, St Paul, MN (US); Cory P. Wright, Woodbury, MN (US)

(21) Appl. No.: 19/658,036

(22) Filed: Apr. 24, 2026

Related U.S. Application Data
(60) Provisional application No. 63/794,238, filed on Apr. 24, 2025.

(51) Int. Cl. A61B 5/283; A61B 5/00; A61B 5/282

(52) U.S. Cl. CPC A61B 5/283; A61B 5/282; A61B 5/6856

(57) ABSTRACT

A catheter for mapping cardiac tissue, the catheter comprising an elongate catheter shaft and an electrode assembly. The catheter shaft has a proximal portion and a shaft distal end, and defines a longitudinal axis of the catheter. The electrode assembly is coupled to the shaft distal end, and comprises a plurality of splines arranged in a semi-circular arc extending partially around the longitudinal axis, the plurality of splines configured to form a plurality of loops extending distally and radially outward from the shaft distal end, and a plurality of spline electrodes disposed along each spline.

### CROSS REFERENCE TO RELATED APPLICATIONS

This application claims priority to U.S. Provisional Application No. 63/794,238 entitled “ELECTROPHYSIOLOGY CATHETER,” filed Apr. 24, 2025, which is herein incorporated by reference in its entirety.

### TECHNICAL FIELD

The present disclosure relates to medical systems and methods for mapping tissue in a patient. More specifically, the present disclosure relates to medical systems and methods for mapping cardiac tissue.

### BACKGROUND

Procedures for treating cardiac arrhythmias (e.g., via thermal or non-thermal ablation therapies) are often preceded, and in some cases followed, by electroanatomical mapping of the cardiac tissue, whereby electrical activity within the heart is sensed and measured by advancing a catheter containing one or more electrodes into the heart, and acquiring data at multiple points within the heart. In such procedures, the positions of the electrodes are tracked via a position tracking system, such that a detailed three-dimensional anatomical map of electrical activity within the heart can be obtained. While a number of electroanatomical mapping catheters have been utilized, there is a continuing need for improved mapping catheter configurations.

### SUMMARY

In Example 1, a catheter for mapping cardiac tissue, the catheter comprising an elongate catheter shaft and an electrode assembly. The catheter shaft has a proximal portion and a shaft distal end, and defines a longitudinal axis of the catheter. The electrode assembly is coupled to the shaft distal end, and comprises a plurality of splines arranged in a semi-circular arc extending partially around the longitudinal axis, the plurality of splines configured to form a plurality of loops extending distally and radially outward from the shaft distal end, and a plurality of spline electrodes disposed along each spline.

In Example 2, the catheter of Example 1, wherein the electrode assembly is coupled to the shaft solely at the shaft distal end.

In Example 3, the catheter of either of Examples 1 or 2, wherein each loop has first and second ends originating at the shaft distal end.

In Example 4, the catheter of any of Examples 1-3, wherein each loop is formed from a single spline.

In Example 5, the catheter of Example 4, wherein a portion of each spline extends through the loop formed by an adjacent spline.

In Example 6, the catheter of either of Examples 4 or 5, wherein each spline is coupled to an adjacent spline along at least a portion thereof.

In Example 7, the catheter of any of Examples 1-3, wherein each loop shares at least one spline with an adjacent loop.

In Example 8, the catheter of Example 7, wherein each loop is formed by a pair of adjacent splines each having a proximal end and an opposite distal end, and a connecting segment extending between the distal ends of thereof.

In Example 9, the catheter of Example 8, wherein each spline is generally straight between the proximal and distal end thereof.

In Example 10, the catheter of Example 8, wherein each spline has a pre-formed curvature between the proximal and distal end thereof.

In Example 11, the catheter of any of Examples 8-10, wherein each connecting segment is canted or curved radially inward toward the longitudinal axis relative to the corresponding splines.

In Example 12, the catheter of any of Examples 1-11, wherein the spline electrodes are equally spaced along each spline.

In Example 13, the catheter of any of Examples 1-11, wherein a spacing between spline electrodes along each spline varies from spline to spline.

In Example 14, the catheter of any of Examples 1-12, wherein the pair of splines forming each loop diverge in the proximal-to-distal direction of the loop.

In Example 15, the catheter of any of Examples 1-12, wherein each spline includes an internal support member and a polymeric covering over the support member.

In Example 16, a catheter for mapping cardiac tissue, the catheter comprising an elongate catheter shaft and an electrode assembly. The catheter shaft has a proximal portion and a shaft distal end, and defines a longitudinal axis of catheter. The electrode assembly is coupled to the shaft distal end, and includes a plurality of splines extending longitudinally and radially outward from the shaft distal end and arranged in an arc around the longitudinal axis, wherein adjacent splines form a respective spline pair, and wherein a spacing between adjacent splines increases in a proximal-to-distal direction along the splines, a plurality of connecting segments connecting distal ends of adjacent splines such that each pair of adjacent splines forms a loop, and a plurality of spline electrodes distributed along each spline.

In Example 17, the catheter of Example 16, wherein each loop shares at least one spline with an adjacent loop.

In Example 18, the catheter of Example 17, wherein each spline is generally straight between the proximal and distal end thereof.

In Example 19, the catheter of Example 18, wherein each connecting segment is canted or curved radially inward toward the longitudinal axis relative to the corresponding splines.

In Example 20, the catheter of Example 19, wherein the electrode assembly further comprises a tip electrode disposed on each connecting segment.

In Example 21, the catheter of Example 20, wherein the spline electrodes are equally spaced along each spline.

In Example 22, the catheter of Example 20, wherein a spacing between spline electrodes along each spline varies from spline to spline.

In Example 23, the catheter of Example 20, wherein each spline includes an internal support member and a polymeric covering over the support member.

In Example 24, the catheter of Example 23, wherein the internal support member is formed from a single piece of shape memory material.

In Example 25, the catheter of Example 17, wherein each spline has a pre-formed curvature between the proximal and distal end thereof.

In Example 26, the catheter of Example 25, wherein the electrode assembly further comprises a tip electrode disposed on each connecting segment

In Example 27, a catheter for mapping cardiac tissue, the catheter comprising an elongate catheter shaft having a proximal portion and a shaft distal end, the catheter shaft defining a longitudinal axis of catheter, and an electrode assembly coupled solely to the shaft distal end. The electrode assembly includes a plurality of splines each having a proximal end and an opposite distal end, the proximal ends arranged in an arc extending partially around the longitudinal axis, each spline extending longitudinally and radially outward from the shaft distal end in a direction transverse to the longitudinal axis, wherein a spacing between the proximal ends of adjacent splines is greater than a spacing between the distal ends thereof, a plurality of connecting segments connecting the distal ends of adjacent splines, and a plurality of electrodes distributed along each spline.

In Example 28, the catheter of Example 27, wherein the electrode assembly further comprises a tip electrode disposed on each connecting segment.

In Example 29, the catheter of Example 28, wherein each spline is generally straight between the proximal and distal end thereof.

In Example 30, the catheter of Example 29, wherein each connecting segment is canted or curved radially inward toward the longitudinal axis relative to the corresponding splines.

In Example 31, the catheter of Example 28, wherein each spline has a pre-formed curvature between the proximal and distal end thereof.

In Example 32, a catheter for mapping cardiac tissue, the catheter comprising an elongate catheter shaft having a proximal portion and a shaft distal end, the catheter shaft defining a longitudinal axis of the catheter, and an electrode assembly coupled solely to the shaft distal end, the electrode assembly comprising a plurality of splines arranged in a semi-circular arc extending partially around the longitudinal axis, each spline of the plurality of splines forming a loop extending distally and radially outward from the shaft distal end, and a plurality of spline electrodes disposed along each spline.

In Example 33, the catheter of Example 32, wherein the plurality of spline electrodes includes a tip electrode located at a distal-most position on each loop.

In Example 34, the catheter of Example 32, wherein a portion of each spline extends through the loop formed by an adjacent spline.

In Example 35, the catheter of Example 34, wherein each spline is coupled to an adjacent spline along at least a portion thereof.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a diagram illustrating an exemplary clinical setting for treating a patient, and for diagnosing and/or treating a heart of the patient, using an electrophysiology system, in accordance with embodiments of the subject matter of the disclosure.

FIG. 2A is a perspective illustration of a distal portion of a splined mapping catheter for use in the electrophysiology system of FIG. 1, in accordance with embodiments of the subject matter of the disclosure.

FIG. 2B is a top view of the distal portion of the catheter of FIG. 2A, in accordance with embodiments of the subject matter of the present disclosure.

FIGS. 2C and 2D are side and end views, respectively, of the distal portion of the catheter of FIG. 2A when viewed along a longitudinal axis 212 thereof, in accordance with embodiments of the subject matter of the present disclosure.

FIGS. 2E and 2F are side and detail views, respectively, of the distal portion of the catheter of FIG. 2A, in accordance with embodiments of the subject matter of the present disclosure.

FIG. 3 is an illustration of an internal support frame of an electrode assembly of the catheter of FIGS. 2A-2F, in accordance with embodiments of the present disclosure.

FIGS. 4A and 4B are perspective and end views of a distal portion of an alternative mapping catheter, in accordance with embodiments of the present disclosure.

FIGS. 5A and 5B are top and side views of a distal portion of an alternative mapping catheter, in accordance with embodiments of the present disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

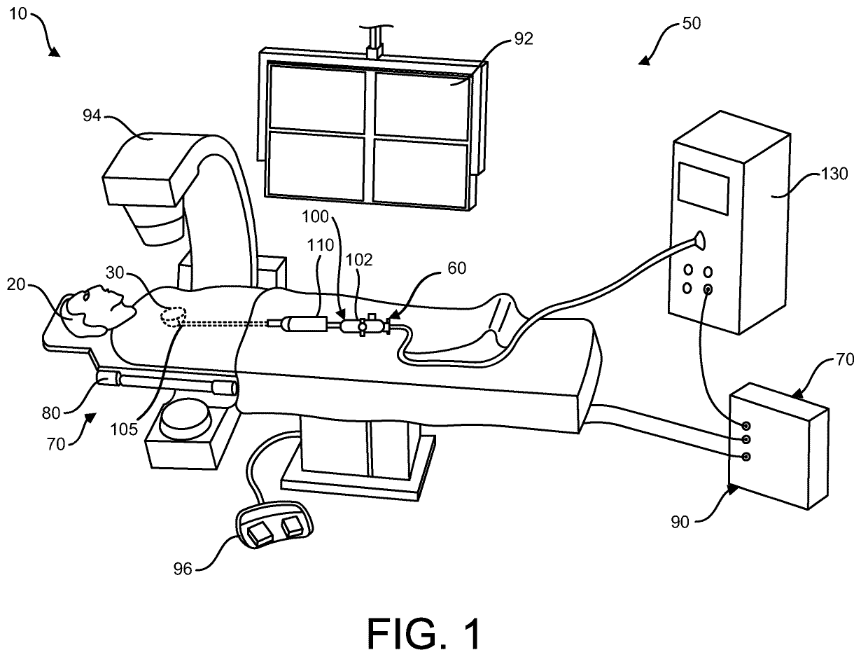

FIG. 1 is a diagram illustrating an exemplary clinical setting 10 for treating a patient 20, and for treating a heart 30 of the patient 20, using an electrophysiology system 50, in accordance with embodiments of the subject matter of the disclosure. The electrophysiology system 50 includes a catheter system 60 and an electro-anatomical mapping (EAM) system 70, which includes a localization field generator 80, a mapping and navigation controller 90, and a graphical display 92. Also, the clinical setting 10 includes additional equipment such as imaging equipment 94 (represented by the C-arm) and various controller elements, such as a foot controller 96, configured to allow an operator to control various aspects of the electrophysiology system 50. As will be appreciated by the skilled artisan, the clinical setting 10 may have other components and arrangements of components that are not shown in FIG. 1.

The catheter system 60 includes an electrophysiology catheter 100 having a proximal portion including a proximal handle 102 and a distal portion 105, an introducer sheath 110, and an ablation console 130. Additionally, the catheter system 60 includes various connecting elements, e.g., cables, umbilicals, and the like, that operate to functionally connect the components of the catheter system 60 to one another and to the components of the EAM system 70. This arrangement of connecting elements is not of critical importance to the present disclosure, and the skilled artisan will recognize that the various components described herein can be interconnected in a variety of ways. By way of example, in various embodiments, the ablation console 130 may be omitted and the electrophysiology catheter 100 may be connected directly to the EAM system 70, e.g., when only electroanatomical mapping procedures are performed.

In embodiments, the introducer sheath 110 is operable to provide a delivery conduit through which the ablation catheter 100, in particular all or part of the distal portion 105 thereof, can be deployed to the specific target sites within the patient's heart 30.

In embodiments, the ablation catheter system 60 is configured to deliver ablative energy to target tissue within the patient's heart 30 to treat arrythmias such as atrial fibrillation or atrial flutter. In embodiments, the ablative energy may take the form of radiofrequency energy or electric field energy to targeted tissue in the patient's heart 30 to create tissue apoptosis, rendering the tissue incapable of conducting electrical signals.

When present, the ablation console 130 is configured to control functional aspects of the catheter system 60. In embodiments, the ablation console 130 includes one or more controllers, microprocessors, and/or computers that execute code out of memory to control and/or perform the functional aspects of the catheter system 60. In embodiments, the memory can be part of the one or more controllers, microprocessors, and/or computers, and/or part of memory capacity accessible through a network, such as the world wide web. In embodiments, the ablation console 130 includes pulse generator hardware, software and/or firmware configure to generate electrical pulses in predefined waveforms, which are transmitted to electrodes on the electrophysiology catheter 100 to generate electric fields sufficient to achieve the desired clinical effect, in particular ablation of target tissue through irreversible electroporation.

The EAM system 70 is operable to track the location of the various functional components of the catheter system 60, and to generate high-fidelity three-dimensional anatomical and electro-anatomical maps of the cardiac chambers of interest. In embodiments, the EAM system 70 can be the OPAL HDx™ mapping system marketed by Boston Scientific Corporation. Also, in embodiments, the mapping and navigation controller 90 of the EAM system 70 includes one or more controllers, microprocessors, and/or computers that execute code out of memory to control and/or perform functional aspects of the EAM system 70, where the memory, in embodiments, can be part of the one or more controllers, microprocessors, and/or computers, and/or part of memory capacity accessible through a network, such as the world wide web.

As will be appreciated by the skilled artisan, the depiction of the electrophysiology system 50 shown in FIG. 1 is intended to provide a general overview of the various components of the system 50 and is not in any way intended to imply that the disclosure is limited to any set of components or arrangement of the components. For example, the skilled artisan will readily recognize that additional hardware components, e.g., breakout boxes, workstations, and the like, can and likely will be included in the electrophysiology system 50.

The EAM system 70 generates a localization field, via the field generator 80, to define a localization volume about the heart 30, and one or more location sensors or sensing elements on the tracked device(s), e.g., the electrophysiology catheter 100, generate an output that can be processed by the mapping and navigation controller 90 to track the location of the sensor, and consequently, the corresponding device, within the localization volume. In the illustrated embodiment, the device tracking is accomplished using magnetic tracking techniques, whereby the field generator 80 is a magnetic field generator that generates a magnetic field defining the localization volume, and the location sensors on the tracked devices are magnetic field sensors.

In other embodiments, impedance tracking methodologies may be employed to track the locations of the various devices. In such embodiments, the localization field is an electric field generated, for example, by an external field generator arrangement, e.g., surface electrodes, by intra-body or intra-cardiac devices, e.g., an intracardiac catheter, or both. In these embodiments, the location sensing elements can constitute electrodes on the tracked devices that generate outputs received and processed by the mapping and navigation controller 90 to track the location of the various location sensing electrodes within the localization volume.

In embodiments, the EAM system 70 is equipped for both magnetic and impedance tracking capabilities. In such embodiments, impedance tracking accuracy can, in some instances be enhanced by first creating a map of the electric field induced by the electric field generator within the cardiac chamber of interest using a probe equipped with a magnetic location sensor, as is possible using the aforementioned OPAL HDx™ mapping system.

Regardless of the tracking methodology employed, the EAM system 70 utilizes the location information for the various tracked devices, along with cardiac electrical activity acquired by, for example, the electrophysiology catheter 100 or another catheter or probe equipped with sensing electrodes, to generate, and display via the display 92, detailed three-dimensional geometric anatomical maps or representations of the cardiac chambers as well as electro-anatomical maps in which cardiac electrical activity of interest is superimposed on the geometric anatomical maps. Furthermore, the EAM system 70 can generate a graphical representation of the various tracked devices within the geometric anatomical map and/or the electro-anatomical map.

In the various embodiments, as described in greater detail below, the electrophysiology catheter 100 is a mapping catheter having an electrode assembly that includes a relatively large number of electrodes in a relatively small area, thus providing the capability of generating high-density electroanatomical maps of cardiac activity.

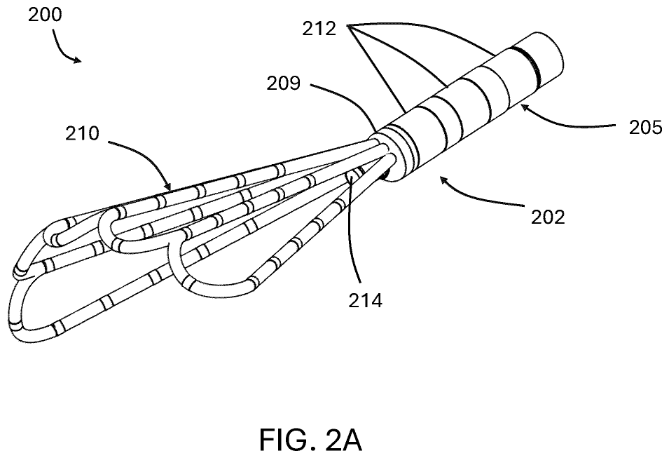

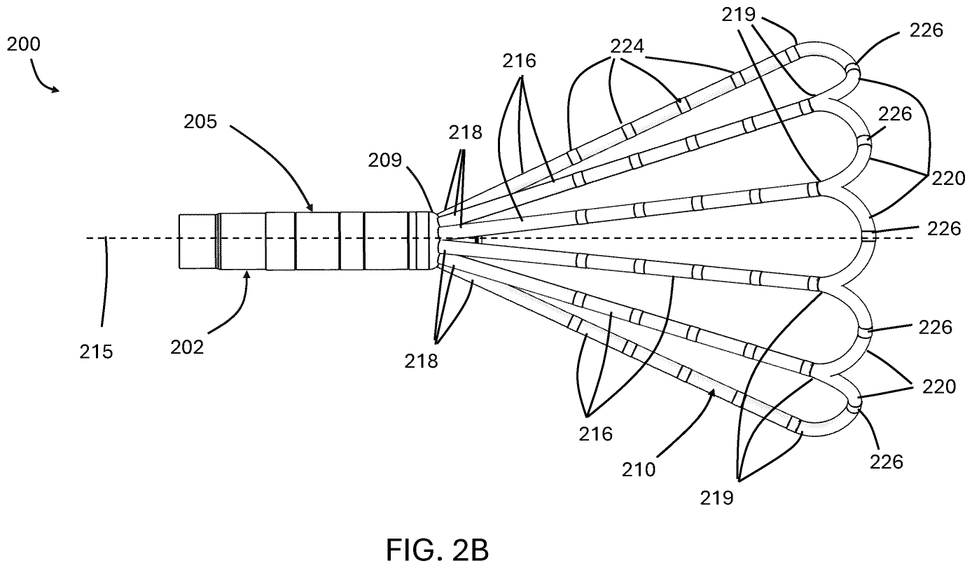

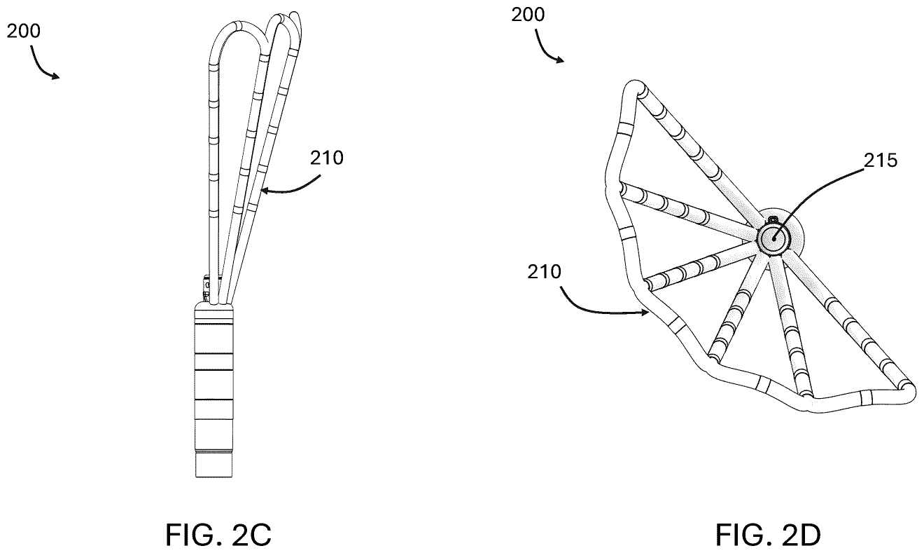

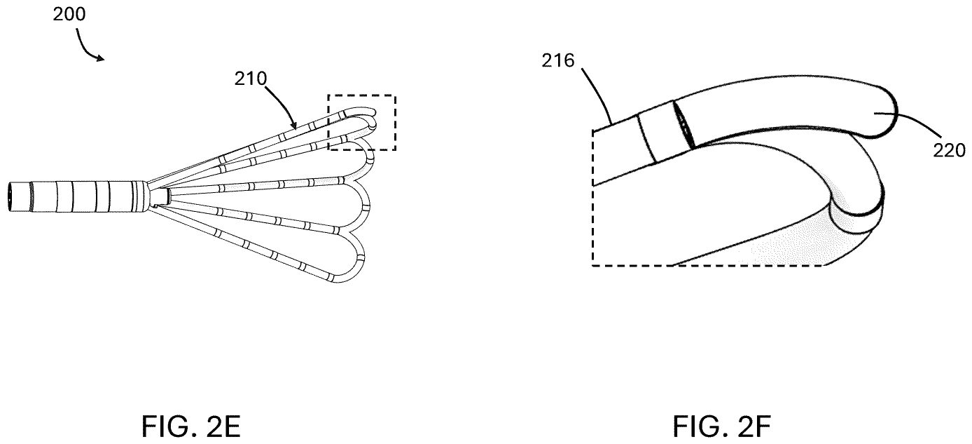

FIG. 2A is a perspective view illustration of a portion of a mapping catheter 200 according to having a catheter distal portion 202 according to an embodiment of the present disclosure. In embodiments, the mapping catheter 200 may correspond to the electrophysiology catheter 100 described with respect to FIG. 1. As shown, the mapping catheter 200 has a tubular catheter shaft 205 having a shaft distal end 209, and an electrode assembly 210 extending distally from the distal end 209 of the catheter shaft 205. In the illustrated embodiment, the mapping catheter 200 further includes shaft electrodes 212 disposed on the shaft 205 proximally of the distal end 209, and a central post 214 extending distally of the shaft distal end 209. When present, the central post 214 may provide additional functionality, including for example, housing or supporting a unipolar reference electrode and/or a magnetic position sensor for use with the EAM system 70 (FIG. 1).

In embodiments, the electrode assembly 210 comprises a flexible framework that is configured to self-expand from a collapsed configuration when constrained within a delivery sheath to a pre-defined expanded configuration as depicted in FIG. 2A. As will be explained in greater detail herein, the electrode assembly 210 comprises a plurality of mapping and sensing electrodes configured for, among other things, sensing cardiac electrical signals, localization of the electrode assembly 210 within the patient anatomy (e.g., via the EAM system 70 of FIG. 1), and determining proximity to target tissue within the anatomy. Overall, the electrode assembly 210 and other electrode assembly embodiments described herein within the scope of the present disclosure, are designed to enable high fidelity sensing of cardiac tissue, e.g., for lesion or conduction block assessment, tissue contact determinations, and the like.

FIG. 2B is a top view of the catheter distal portion 202, according to one embodiment of the subject matter of the present disclosure. As shown, in the illustrated embodiment, the catheter shaft 205 defines a longitudinal axis 215 of the catheter 200. As further shown, the electrode assembly 210 includes a plurality of splines 216 each having a proximal end 218 and an opposite distal end 219. The electrode assembly 210 further comprises a plurality of connecting segments 220, a plurality of spline electrodes 224, and a plurality of tip electrodes 226. To avoid clutter, the spline electrodes 224 are labeled with respect to only one of the splines 216, but the skilled artisan will readily recognize that the other splines further include spline electrodes disposed thereon.

As shown, the connecting segments 220 extend between and connect the distal ends 219 of adjacent splines 216, such that each pair of adjacent splines 216 form a loop structure, with the ends of each loop originating at the shaft distal end 209, and the respective connecting segment 220 forming a distal end portion of each loop. As can be seen in FIG. 2B, each loop shares at least one spline 216. As further shown, the spline electrodes 224 are spaced along each spline 216 between the proximal and distal ends 218, 219, and one of the tip electrodes 226 is disposed approximately in the center of each connecting segment 220. In some embodiments, the spline electrodes 224 are equally spaced along each spline 216, whereas in other embodiments the spline electrodes 224 may have unequal spacing along each spline 216 (e.g., the spacing between adjacent spline electrodes 224 on a given spline may vary along the length of the spline 216). In some embodiments, the spacing of the spline electrodes 224 along each spline can vary from spline 216 to spline 216. The number of spline electrodes 224 may also vary from spline-to-spline and/or among the various embodiments. Additionally, in some embodiments, some or all of the tip electrodes 226 may be omitted.

FIGS. 2C and 2D are side and end views (when viewed along the longitudinal axis 215 of the catheter 200), respectively, of the catheter distal portion 202, in accordance with embodiments of the subject matter of the present disclosure. Referring collectively to FIGS. 2B-2D, in the illustrated embodiment, the splines 216 are extend longitudinally and radially from the shaft distal end 209 in a direction that is transverse to the longitudinal axis 215. As further shown, the splines 216 are arranged in a semi-circular arc that partially circumscribes the longitudinal axis 215. Additionally, the splines 216 radiate or diverge outward relative to one another in the proximal-to-distal direction, such that the spacing between adjacent splines 216 increases in the proximal-to-distal direction. As shown, in the expanded configuration illustrated in FIGS. 2B-2D, the electrode assembly 210 assumes a fan-like shape that partially circumscribes the longitudinal axis 215. Accordingly, the spline-to-spline spacing of spline electrodes 224 also increases in the proximal-to-distal direction. In the illustrated embodiment, the splines 216 are arranged in an arc that extends approximately half-way about the longitudinal axis 215, whereas in other embodiments the corresponding arc may be longer or shorter (e.g., by providing more or fewer splines 216 or by increasing the spline spacing at the shaft distal end 209).

FIGS. 2E and 2F are side and detail views, respectively, of the catheter distal portion 202, in accordance with embodiments of the subject matter of the present disclosure. As shown, and in particular in FIG. 2F the connecting segments 220 curve or are canted radially inward (i.e., toward the longitudinal axis 215) relative to the splines 216. In this manner, the connecting segments 220 assume a ski-tip like configuration. In other embodiments, the connecting segments 220 are substantially in line with the splines 216 that they connect.

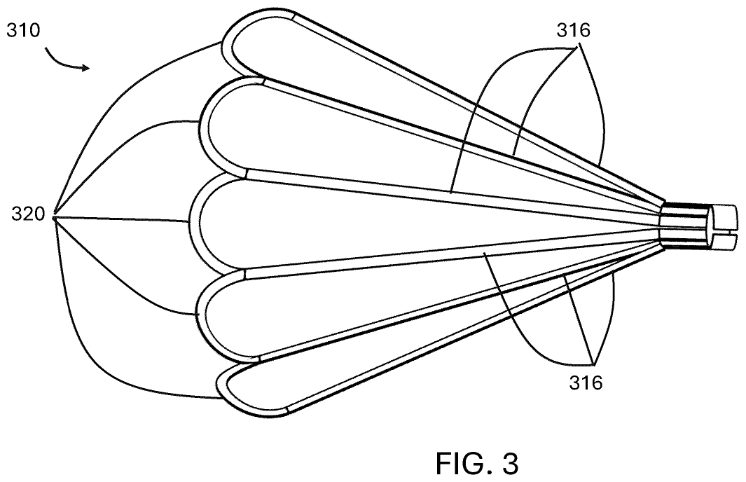

In embodiments, the electrode assembly 210 is constructed of an internal support framework covered by a tubing or overmolded covering made of a biocompatible material, e.g., a polyether block amide or other similar material commonly used in electrophysiology catheters, and the spline and tip electrodes 224, 226 are disposed on this outer covering. FIG. 3 is a bottom view of an exemplary internal frame 310 that could be used in the construction of the electrode assembly 210 of FIG. 2A. As shown, the frame 310 includes spline supports 316 and connecting segment supports 320 integrally formed in the frame 310. In one embodiment, the frame 310 may be formed from a single piece of tubular material, e.g., by laser cutting or other conventional manufacturing techniques. In embodiments, the frame 310 may not include the connecting segment supports 320. In such embodiments, the mechanical properties of the connecting segments may be provided by proper selection of the aforementioned covering material. In various embodiments, the frame 310 may be made from a material having shape memory properties, e.g., nitinol, which can advantageously be employed to define the desired expanded shape of the electrode assembly.

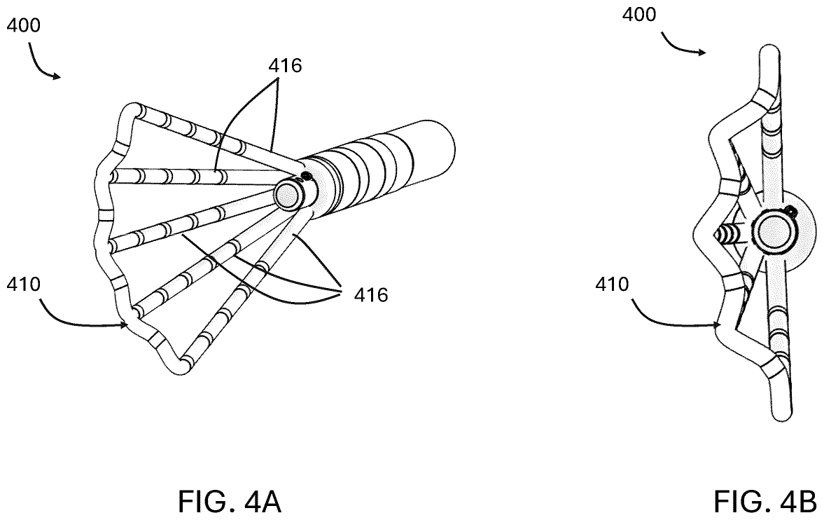

FIGS. 4A and 4B are perspective and end views, respectively of a distal portion 405 of an alternative mapping catheter 400 having an electrode assembly 410, in accordance with embodiments of the present disclosure. The electrode assembly 410 is in most respect similar to the electrode assembly 210 described above, but differs in that it has fewer splines 416, and the amount of spline-to-spline divergence in the proximal-to-distal direction of the electrode assembly 410 is less than that of the electrode assembly 210. As the skilled artisan will appreciate based on the disclosure, the fan-like electrode configurations disclosed herein can be tailored to provide a wide range of form factors and corresponding functionalities.

In the illustrated embodiments, the splines (e.g., splines 216, 416) are generally or substantially straight between their respective proximal and distal ends. However, in some embodiments, the splines 216, 416 may be designed with a pre-formed curvature to impart an overall curved or spoon-like shape to the electrode assembly 210, 410 when expanded (e.g., when the pre-formed curvature is concave from the perspective of the longitudinal axis).

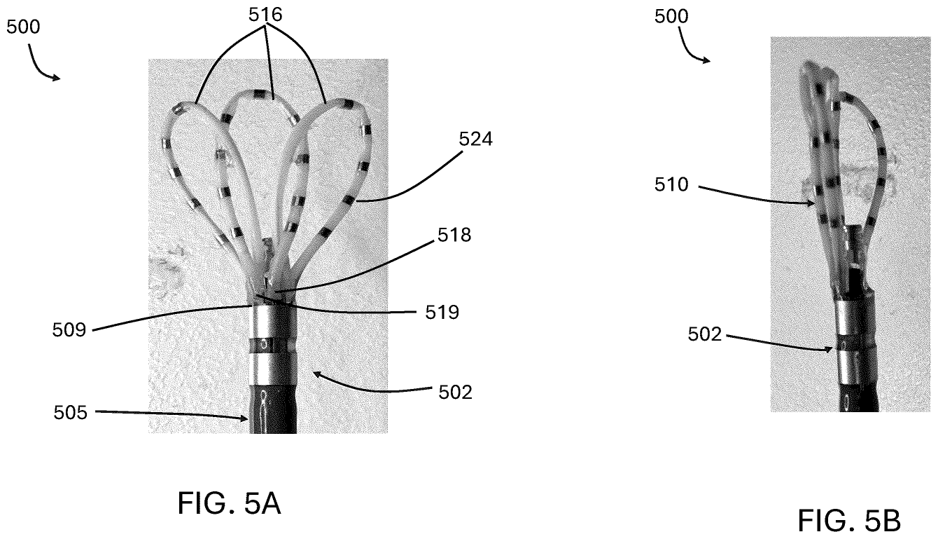

FIGS. 5A and 5B are top and side views, respectively, of a distal portion 502 of a mapping catheter 500 in accordance with an alternative embodiment of the subject matter of the disclosure. With reference to FIGS. 5A and 5B collectively, as shown, the catheter 500 includes a catheter shaft 505 having a distal end 509, and an electrode assembly 510 includes a plurality of splines 516 each having opposing spline ends 518, 519 that extend from the shaft distal end 509 to form a loop (for clarity of illustration, these features are labeled on only one spline in FIG. 5A). As further shown, a plurality of spline electrodes 524 are disposed along a length of each spline 516. Additionally, the adjacent splines 516 are interweaved, in that a portion of each spline extends through the loop formed by an adjacent spline to facilitate formation of the fan-like configuration shown in FIGS. 5A and 5B. As with the electrode assemblies 210, 410, the distal-most portion of each loop may be curved or canted inwardly relative to the more proximal portions of the curve to form a “ski-tip” like distal end portion of the loop. In embodiments, the splines 516 may be configured so that the major length of each loop extends substantially linearly from the shaft distal end 509 (except in the case the loop has a canted distal portion as described above), or alternatively, so that each loop has a concave curvature as described above in connection with the electrode assembly 210. As can be seen in FIG. 5B, as with the other embodiments described herein, the electrode assembly 510 is arranged in a partial arc around a portion of the center of the catheter shaft 505. In some embodiments, adjacent spline loops may be secured together (e.g., by adhesive or a coupling element) at one or more locations along the lengths of the corresponding splines 516 to control the shape and or deformation of the electrode assembly 510 under external forces.

The various embodiments of electrode assemblies described herein have a number of advantages. Because the various electrode assemblies are coupled to the catheter shaft solely at the shaft distal end, the electrode assembly exhibits a high degree of controlled flexibility. Additionally, the fan-like electrode assembly configuration provides for relatively fast mapping and wide anatomical coverage using the distally-located electrodes (which have relatively large spline-to-spline electrode spacing and thus form relatively wide bipoles), while at the same time providing a high electrode density near the distal end of the catheter shaft (where the catheter exhibits the greatest degree of control and stability). Also, in embodiments, the catheters 200, 400, 500 may be configured as deflectable catheters, whereby the user can change the curvature of the corresponding catheter shaft by operation of an actuator on the catheter handle. In such embodiments, it may be advantageous to orient the widest dimension of the electrode assembly (e.g., near its distal end) perpendicular to the plane of catheter deflection to enable the clinician to impart a sweeping motion to the electrode assembly by operation of the actuator. Additionally, when the catheter includes an indifferent/reference electrode disposed on the post 214 (see FIG. 2A) or some other location near the spline proximal ends, relatively large uni-pole separation utilizing the more distal spline electrodes and/or the tip electrodes can be accomplished, while ag the same time forming near-field uni-poles with the more densely spaced proximal spline electrodes.

With further reference to the catheters 200, 400 and 500, the “ski-tip” configuration of the allows for relatively rapid side-to-side and forward (when the tip electrodes are present) mapping of cardiac walls without catching on internal structures. Additionally, the curved connecting segments enhance collapsibility of the electrode assembly with forward movement for navigation into relatively confined spaces such as the pulmonary veins (i.e., the ski-tip configuration of the connecting segments facilitates collapse of the electrode structure when pressed against the ostium of the pulmonary vein). Additionally, the arc-shaped fan configurations described herein provide enhanced ability to map curves structures such as the pulmonary vein ostia as compared to planar array type catheter configurations.

The skilled artisan will readily appreciate that any of the electrode assemblies 210, 410 and 510 described herein can also be constructed utilizing flexible printed circuit (FPC) techniques. In such embodiments, the splines/connecting segments may be formed with a dielectric substrate (which may or may not be disposed over a structural support, and the various electrodes and electrical traces may be formed on the substrate using conventional FPC design and manufacturing techniques.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A catheter for mapping cardiac tissue, the catheter comprising:
an elongate catheter shaft having a proximal portion and a shaft distal end, the catheter shaft defining a longitudinal axis of catheter; and
an electrode assembly coupled to the shaft distal end, the electrode assembly including:
a plurality of splines extending longitudinally and radially outward from the shaft distal end and arranged in an arc around the longitudinal axis, wherein adjacent splines form a respective spline pair, and wherein a spacing between adjacent splines increases in a proximal-to-distal direction along the splines;
a plurality of connecting segments connecting distal ends of adjacent splines such that each pair of adjacent splines forms a loop; and
a plurality of spline electrodes distributed along each spline.

2. The catheter of claim 1, wherein each loop shares at least one spline with an adjacent loop.

3. The catheter of claim 2, wherein each spline is generally straight between the proximal and distal end thereof.

4. The catheter of claim 3, wherein each connecting segment is canted or curved radially inward toward the longitudinal axis relative to the corresponding splines.

5. The catheter of claim 3, wherein the electrode assembly further comprises a tip electrode disposed on each connecting segment.

6. The catheter of claim 5, wherein the spline electrodes are equally spaced along each spline.

7. The catheter of claim 5, wherein a spacing between spline electrodes along each spline varies from spline to spline.

8. The catheter of claim 5, wherein each spline includes an internal support member and a polymeric covering over the support member.

9. The catheter of claim 8, wherein the internal support member is formed from a single piece of shape memory material.

10. The catheter of claim 2, wherein each spline has a pre-formed curvature between the proximal and distal end thereof.

11. The catheter of claim 10, wherein the electrode assembly further comprises a tip electrode disposed on each connecting segment.

12. A catheter for mapping cardiac tissue, the catheter comprising:
an elongate catheter shaft having a proximal portion and a shaft distal end, the catheter shaft defining a longitudinal axis of catheter; and
an electrode assembly coupled solely to the shaft distal end, the electrode assembly including:
a plurality of splines each having a proximal end and an opposite distal end, the proximal ends arranged in an arc extending partially around the longitudinal axis, each spline extending longitudinally and radially outward from the shaft distal end in a transverse direction relative to the longitudinal axis, wherein a spacing between the proximal ends of adjacent splines is greater than a spacing between the distal ends thereof;
a plurality of connecting segments connecting the distal ends of adjacent splines; and
a plurality of electrodes distributed along each spline.

13. The catheter of claim 12, wherein the electrode assembly further comprises a tip electrode disposed on each connecting segment.

14. The catheter of claim 13, wherein each spline is generally straight between the proximal and distal end thereof.

15. The catheter of claim 14, wherein each connecting segment is canted or curved radially inward toward the longitudinal axis relative to the corresponding splines.

16. The catheter of claim 13, wherein each spline has a pre-formed curvature between the proximal and distal end thereof.

17. A catheter for mapping cardiac tissue, the catheter comprising:
an elongate catheter shaft having a proximal portion and a shaft distal end, the catheter shaft defining a longitudinal axis of the catheter;
an electrode assembly coupled solely to the shaft distal end, the electrode assembly comprising:
a plurality of splines arranged in a semi-circular arc extending partially around the longitudinal axis, each spline of the plurality of splines forming a loop extending distally and radially outward from the shaft distal end; and
a plurality of spline electrodes disposed along each spline.

18. The catheter of claim 17, wherein the plurality of spline electrodes includes a tip electrode located at a distal-most position on each loop.

19. The catheter of claim 17, wherein a portion of each spline extends through the loop formed by an adjacent spline.

20. The catheter of claim 19, wherein each spline is coupled to an adjacent spline along at least a portion thereof.
